(19) United States
(12) Patent Application Publication
(10) Pub. No.: US 2023/0386663 A1
(43) Pub. Date: Nov. 30, 2023

(54) SYSTEM AND METHOD FOR IMPROVING CLINICAL EFFECTIVENESS USING A QUERY REASONING ENGINE

(71) Applicant: Px Health AI Inc, Santa Cruz, CA (US)

(72) Inventor: Michael FOODY, Santa Cruz, CA (US)

(73) Assignee: Px Health AI Inc., Santa Cruz, CA (US)

(21) Appl. No.: 18/200,966

(22) Filed: May 23, 2023

Related U.S. Application Data
(60) Provisional application No. 63/345,076, filed on May 24, 2022.

(51) Int. Cl. G16H 50/20; G16H 15/00

(52) U.S. Cl. CPC G16H 50/20; G16H 15/00

(57) ABSTRACT

A system and method that accepts and responds to the health queries according to one or more computational knowledge graphs (CKGs). The method includes constructing CKGs, loading CKGs into the query reasoning engine, the query reasoning engine: accepting health queries, performing computations according to the specific patient health situation and CKG; returning results and effecting updates. The method includes accessing information and effecting updates in healthcare enterprise software. The method includes summarizing the health situation, history and location within the health flow according to the CKG. The method includes identifying and correcting missing, conflicting or erroneous health information. The method includes means for clinicians of normal medical skill to construct CKGs.

This application claims priority to U.S. Provisional Patent Application No. 63/345,076, filed on May 24, 2022, the disclosures of which are incorporated herein by reference in its entirety as part of the present application.

### TECHNICAL FIELD

The disclosure of this application relates to a system and a method for improving clinical effectiveness using a Query Reasoning Engine, that improves efficiency and quality in healthcare by processing multiple sources of data into a reduced graph of relevant context, chain of thought reasoning and trustworthy data according to a healthcare computational knowledge graph (CKG).

### BACKGROUND

Current systems for documenting interactions with patients are based upon storing data of “electronic health records,” which are often referred to as EHR systems or, in some cases, referred to as “electronic medical records” (EMR) systems. That is, they are “a digital version of a patient's paper chart” for recording and storing individual patients' records of diagnoses, procedures, medications, treatment plans, immunization dates, allergies, radiology images, laboratory test results and the like. But EHRs aren't the only source of data that clinical staff need to consult to build an effective, holistic understanding of the patient's situation. There are a wide variety of data sources involved, including PACS (Picture archiving and communications systems) for imaging like CT scans; CRM (Customer relationship management) systems for patient personal information; PMS (Practice management systems) for billing and insurance; and the like (collectively referred to as “healthcare enterprise systems”).

EHR systems are widely documented to waste 25-35% of physicians time, in part because they don't understand clinical situations per se. Consequently, physicians are required to “dig through” medical records to gather the information they need for the specific given clinical situation in order to understand the status, history, context and available choices. Moreover, substantial information on the diagnostic and care process itself is lost and can only be partially and inaccurately inferred from an EHR. It's not just individual clinical staff affected. Similar challenges affect clinical teams. Overall, approximately ˜65% of physicians' time is wasted, and approximately half of that is the time wasted individually or in teams to fully understand, share and communicate the patient's health situation using healthcare enterprise systems (although the figure varies on a physician-by-physician basis.)

The types of information required to build a complete understanding of the patient's situation in the minds of clinical staff fall into three categories: clinical, personal and economic. On the clinical front, for clinical staff to build a full understanding of a clinical situation includes: sorting through the volumes of information available to determine the clinical concept values that are specifically relevant to the current situation; constructing the relevant summarized history along with the reasoning for decisions along the way—the chain of thought reasoning path; identifying the current context of where the patient is in the overall path and plan of care; as well as placing what has changed for the patient in the timeline. Specific challenges associated with gathering relevant data from the information in healthcare enterprise systems are widely known: information overload (far too much information); information scatter (information is scattered throughout the records); information underload (information is missing); information conflict (different sources of information conflict); and erroneous information (some information is simply wrong). This creates an additional burden for any automated system—providing trustworthy information.

On the personal front, desired outcomes for patients as well as incentives which will aid in reaching them can be highly individual, for example, a patient might say “after the operation, I just want to be able to dance at my daughter's wedding.” Incorporating such information into the clinicians' understanding of the situation has demonstrable value.

On the economic front, determining medical economics and insurance coverage throughout the care process can be notoriously complex. Medical procedures may be covered in some circumstances but not others; coverage may involve subjective decisions on medical necessity, the level of payment may depend on the definition of the complexity of the medical case; decisions to treat may depend upon cost effectiveness; and so on.

Knowledge graphs have become popular as a modern means to implement an information model. “Medical knowledge graphs” (MKGs) are well known as a means to describe medical knowledge, including terminology, conceptual relationships, ontology, probabilities of occurrence and the like—in great detail and breadth. Similarly, “personal knowledge graphs” (PKGs), are well known as an underlying mechanism for implementing personalization in search, advertising, e-commerce, and entertainment. Further, “economic knowledge graphs” (EKGs) are well known as an underlying mechanism for implementing purchasing behavior tracking and doing economic modelling, they too are more broadly used within proprietary commercial systems.

Creating and updating information models, including knowledge graphs (KGs), in healthcare entails a number of difficult challenges. One is a result of the fact that medicine is so vast and complex. There are 40 specialties, with 160 subspecialties—and they are all highly detailed, which leads to voluminous KGs. Current methods to create and update KGs suffer from some substantial limitations: they're opaque to collaborative review and testing; they're constructed with tools designed for knowledge engineers not regular physicians which substantially limits the number of trained physicians who can create, edit or manage them; there's no effective way to annotate the KG with additional information like amounts, times, costs, etc. Moreover, the rate of medical knowledge generation continues to accelerate, which makes any KG become outdated quickly. In addition, every organization needs its own, customized KG to deal with its specific blend of skills, staff, equipment organization and the like.

To date there have been systems which attempt to aid clinicians by manipulating the information model: prioritizing the information they see; by organizing the information into simplistic models like SOAP or OEAI in timelines and calling it a “cognitive model” or “mental model”; by “normalizing the information” into neutral models like SNOMED or VFusion maps and so on but with limited success. The issue with an information model approach is that it's not actionable—it's not taking action to reason the way different clinicians do as they seek to fully understand patient's health context and situation, to concisely communicate it to other clinicians within and across teams, and to answer questions about it.

Systems today can't answer the type of questions that will save the most time for clinicians and deliver the best outcomes for patients, given what matters most to them—questions like “Summarize the case for me.”, “Why was a TURBT ruled out?”, “What decisions are coming up over the next 24 hours,” “Will his last appointment be before his daughter's wedding?” “What are the patient's out-of-pocket costs for each of our options?”

More recently there have been attempts to solve the problem using a chat user interface of a large language model. Large language models represent an alternative to KGs for creating a compendium of knowledge with the ability to answer questions about it. While they can handle the “language portion” of the questions above, they are well known to lack the ability to provide accurate context, chain of thought reasoning and trustworthy data.

### SUMMARY

Disclosed herein is a system for improving clinical effectiveness, which comprises 1) a Query Reasoning Engine; 2) sources of health queries and updates, which may include various operative applications with a graphical user interface, a natural language model chat system, or other operative applications; 3) a module for creating computational knowledge graphs (CKGs) which may incorporate a combination of clinical, personal and economic knowledge, and wherein the module may include software applications that, in a preferred embodiment are designed for use by clinicians of normal skill in the medical arts; 4) external health condenser modules; and 5) a knowledge storage system (KSS) to store CKGs, the “path walked through the CKG, and validated information, and the like. In a preferred embodiment each aforesaid individual component is implemented in a cloud system, and it can be understood that consequently each of the individual components may comprise multiple communicating computers and data storage elements so as to distribute computing tasks and computing load.

Also disclosed herein is a method of improving clinical effectiveness comprises the steps of 1) constructing computational knowledge graphs (CKGs), whereupon the Query Reasoning Engine may load one or more CKGs which may also be stored in the KSS for further use; 2) the Query Reasoning Engine may accept health queries and updates that are transmitted to it from applications that have a graphical user interface, from chatbots utilizing natural language models, from autonomous external applications and the like; and for health queries 3) the Query Reasoning Engine utilizing the CKG in the process of executing the health query, performing calculations according to the specific patient, situation and relevant portion of the CKG, then returning health query results that consist of the concise minimum required context, chain of thought reasoning and validated trustworthy health concept information and structure necessary to understand the situation, or for teams to maintain their shared understanding as it changes state over time; 4) the applications that source and transmit the queries to the reasoning engine then formatting the query response to communicate them to users in an efficient manner, graphical or otherwise, or further processing them according to the application. For health queries that incorporate updates or mutations, 5) the Query Reasoning Engine performing calculations according to the specific patient, situation and CKG and to deconstruct the updates so as to effect them in the external software systems; and 6) the Query Reasoning Engine storing, as determined by the CKG, portions of query responses updates, and mutations including new condensers, health situations and flows, as a case history graph in the KSS for later re-use.

Also disclosed herein is a method of improving clinical effectiveness comprises the steps of 1) constructing computational knowledge graphs (CKGs), whereupon the Query Reasoning Engine may load one or more CKG and may store them in the KSS for further use; 2) the Query Reasoning Engine may accept health queries and updates that are transmitted to it from software systems that have any one of: a graphical user interface, a chatbot interface utilizing natural language models, or an audio interface; or from software systems that have no use interaction, or software systems where the query was not triggered by direct user interaction and the like; and for health queries 3) the Query Reasoning Engine utilizing the CKG, in the process of executing the health query, to perform calculations according to the specific patient and health situation, then returning health query responses that consist of the concise minimum required context, chain of thought reasoning and validated trustworthy health concept information and structure necessary to understand the situation, or for teams to maintain their shared understanding as it changes state over time; and 6) the applications that source and transmit the queries to the reasoning engine then formatting the query response to communicate them to users in an efficient manner, graphical or otherwise, or further processing them according to the application. For health queries that incorporate updates or mutations, 7) the Query Reasoning Engine performing calculations according to the specific patient, situation and CKG, and deconstructing the updates and mutations so as to effect updates and mutations in external software systems.

Step 3) above further comprises the steps of a) using the Determination Engine to determine from the patient context and the CKG the health situation, the health context, and the location within the overall health flow; b) using the Determination Engine according to the CKG to determine the minimum matching graph of condensers which are subsequently passed to the Condenser Engine; c) the Condenser Engine recursively determines the list of information that needs to be pulled from external systems and creating the graph, along with the corresponding valuator, d) the graph and valuators being passed to the Transformation Engine or Dynamic Calling Engine for retrieval; e) the Condenser Engine's Resolver resolving downward and condensing upwards the health concepts utilizing the KSS, internal or external condenser modules, functions, reasoners or data according to the CKG. As it does so, storing the determinative factors from each health concept calculation in the KSS; and f) the Condenser Engine condensing and including in its query response any requested chain of thought reasoning (including the determinative factors), potential actions as appropriate and context.

Also provided herein is a method of producing validated, trustworthy health concept values from enterprise healthcare systems when queried to do so, wherein during step 3.c) above, further comprising the steps of i) automatically identifying for each condensation step when underlying concepts are missing, conflicting, or containing erroneous values; ii) constructing a response graph containing these results; iii) an external application enabling end-users via a graphical user interface or automated systems navigate the Condenser Graph to reach the error locations identified in the error response graph; iv) the end-users or automated system verifying or correcting results as necessary; and iv) storing corrected results in the KSS and updating the CKG so in future Condensers and Valuators access the corrected results.

It can be understood that resolvers may be updated overtime to utilize different condensers, from among manual (user driven) condensers, function condensers, rule-based condensers, machine learning-based condensers, condensers generated by the aforementioned CKG construction method and the like.

Further provided herein is a method facilitate economic reasoning, comprising the steps of 1) transferring the CKG and state to external software; 2) the external software annotating the CKG with additional economic considerations (such as patient out of pocket costs, expected future costs, RUV and the like), or modifying the CKG (such to add or remove prerequisites or covered branches); and 3) the modified CKG being returned for use in the Query Reasoning Engine, either in total or as the difference; 4) the Query Reasoning Engine notifying and updating applications of the changes for display to users; and 5) transferring to the external software the path followed, according to the modified CKG for validation.

Still further provided is a method of constructing CKGs wherein different clinicians of normal skill in the medical arts can construct portions of the CKG, the portions which later can be combined to create full CKGs, comprising the steps of 1) selecting from among familiar source models, which may grow over time but which include guidelines flowcharts, exemplar case presentations, illness scripts, adjacent disease differentiators and the like; 2) optionally importing CKGs constructed by related or unrelated parties; 3) using any of a number of synchronized (bi-directional) tools in any sequence or order to create CKGs, the tools including: written exemplars case examples analyzed by natural language-based tools; graphical flow tools; and graphical user interface tools; and the like 4) annotating and connecting health situations and health concepts; 5) validating and testing CKGs; and 6) optionally exporting CKGs for use by related or unrelated parties.

### BRIEF DESCRIPTION OF DRAWINGS

FIG. 1A shows a schematic drawing of the system according to a preferred embodiment.

FIG. 1B shows a flow diagram of the overall method according to a preferred embodiment.

FIG. 1C shows a schematic drawing of the query reasoning engine according to a preferred embodiment.

FIG. 1D shows a flow diagram of the method of the query reasoning engine.

FIG. 1E shows a hypothetical example of chat with a chatbot that generates queries to, and formats results from the query reasoning engine.

FIG. 1F shows a hypothetical example of automatically constructed graphical user interface according to a health query response from the query reasoning engine.

FIG. 2 shows a flow diagram of the method of the Determination Engine.

FIG. 3 shows a flow diagram of the method of the Condenser Engine.

FIG. 4 shows a schematic drawing of the CKG constructor according to a preferred embodiment.

FIG. 5 shows a flow diagram of the method of the CKG constructor.

FIGS. 6A.1 and 6A.2 show a flow diagram of the methods of the natural language-based tool.

FIGS. 6B.1 and 6B.2 show a flow diagram of the methods of the GUI-based tool.

FIGS. 6C.1 and 6C.2 show a flow diagram of the methods of the Flow tool.

FIG. 7 shows a flow diagram of the method of validating health concept values.

FIG. 8 shows a flow diagram of the method to facilitate economic reasoning.

### DETAILED DESCRIPTION

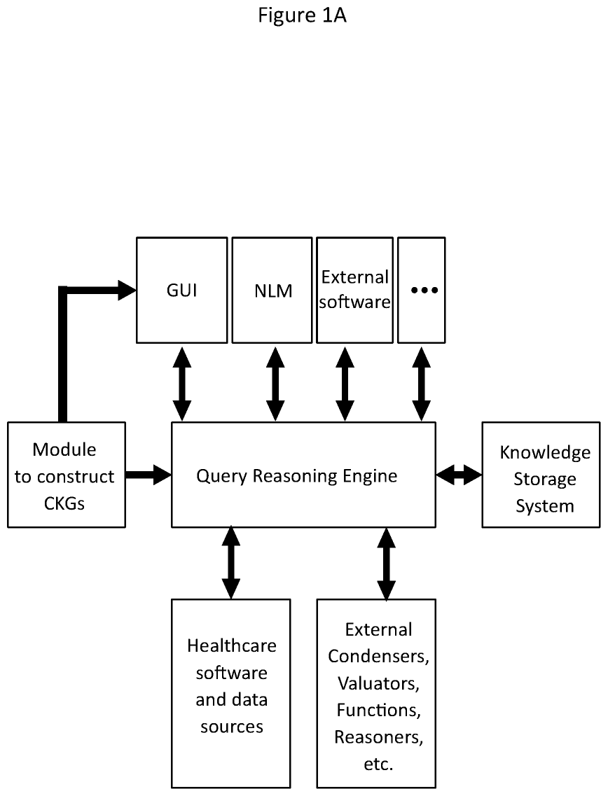

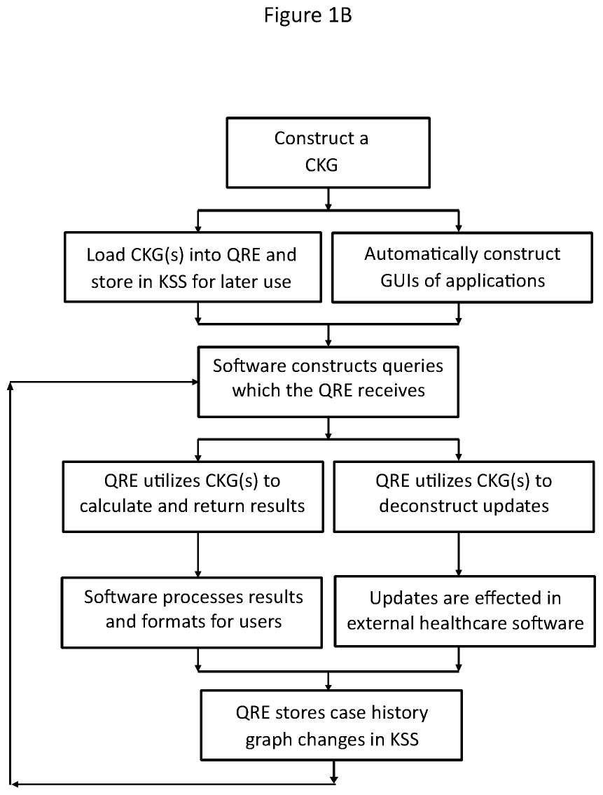

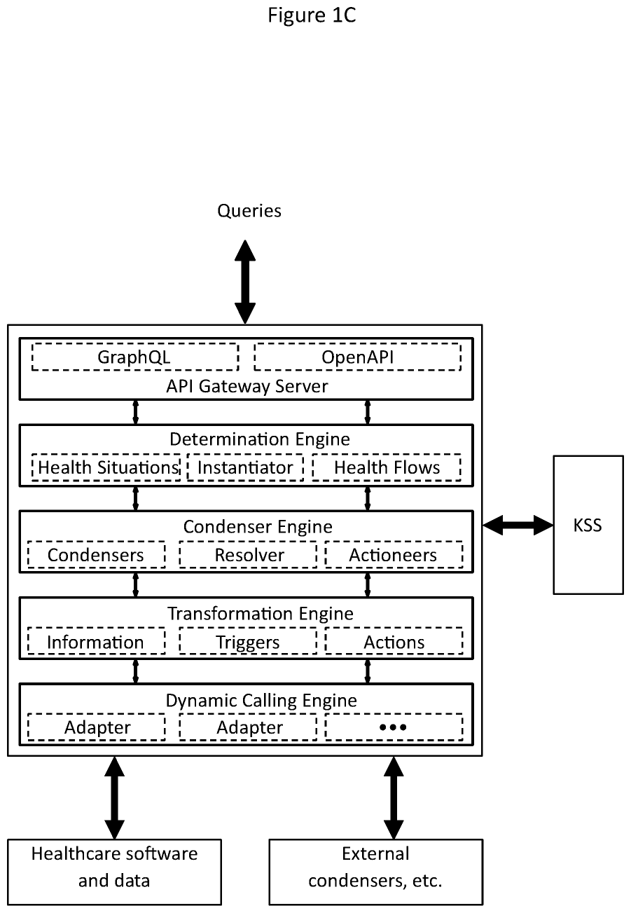

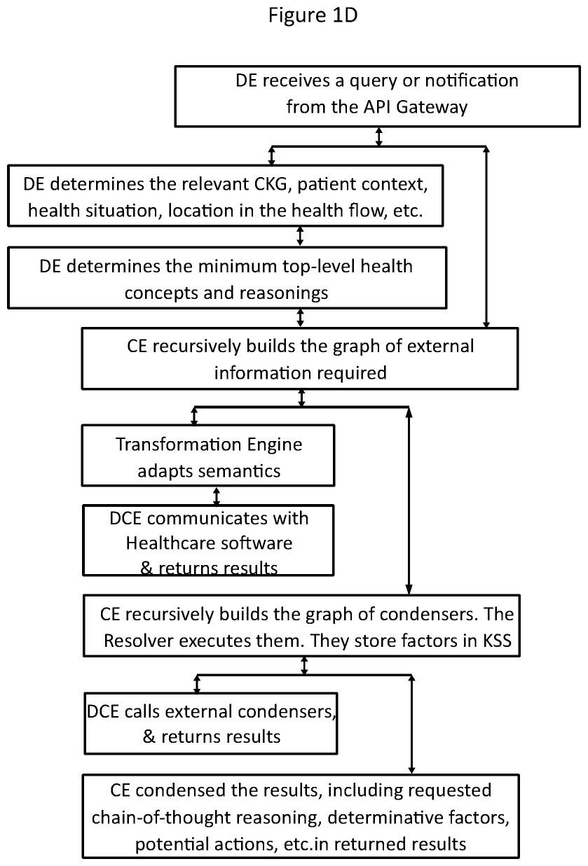

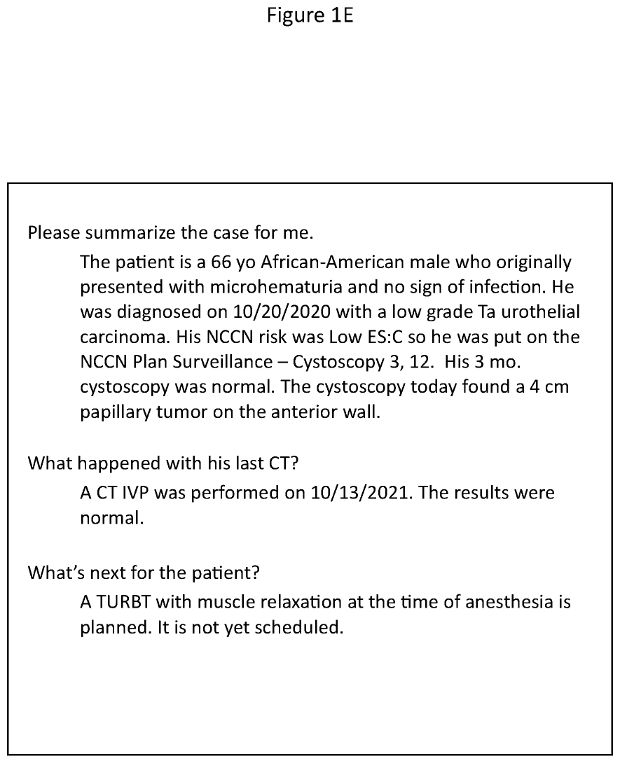

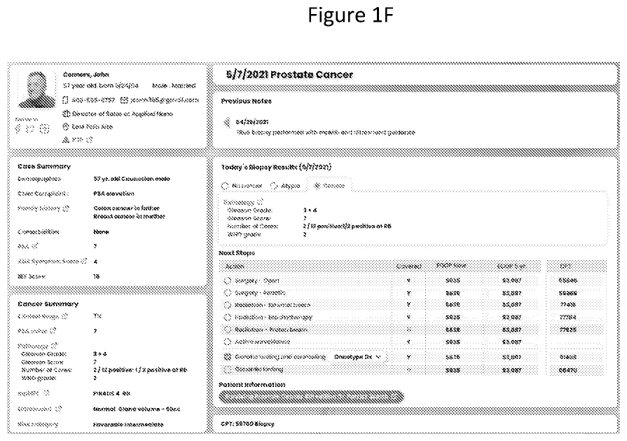

FIG. 1A illustrates a system for improving clinical effectiveness, which comprises 1) a Query Reasoning Engine (QRE); 2) sources of health queries and updates, which may include various operative applications with a graphical user interface (GUI), a natural language model chat system (NLM), or other operative applications; 3) a module for constructing computational knowledge graphs (CKGs) which may incorporate a combination of clinical, personal and economic knowledge, wherein the module may include software applications that, in a preferred embodiment are designed for use by clinicians of normal skill in the medical arts; 4) external health condenser modules; and 5) a knowledge storage system (KSS) to store CKGs, the “path walked through the CKG”, validated information, and the like. In a preferred embodiment each aforesaid individual component is implemented in a cloud system, and it can be understood that consequently each of the individual components may comprise multiple communicating computers and data storage elements so as to distribute computing tasks and computing load.

FIG. 1B illustrates a method of improving clinical effectiveness comprises the steps of 1) constructing computational knowledge graphs (CKGs), whereupon the Query Reasoning Engine may load one or more CKGs which may also be stored in the KSS for further use; 2) the Query Reasoning Engine may accept health queries, which may include updates and mutations, that are constructed by and transmitted to it from software systems that have any one of: a graphical user interface, a chatbot interface utilizing natural language models, or an audio interface; or from software systems that have no use interaction, or software systems where the query was not triggered by direct user interaction and the like; and for health queries 3) the Query Reasoning Engine utilizing the CKG in the process of executing the health query, performing calculations according to the specific patient, situation and relevant portion of the CKG, then returning health query results that consist of the concise minimum required context, chain of thought reasoning and validated trustworthy health concept information and structure necessary to understand the situation, or for teams to maintain their shared understanding as it changes state over time; 4) the applications that source and transmit the queries to the reasoning engine then formatting the query response to communicate them to users in an efficient manner, graphical or otherwise, or further processing them according to the application. For health queries that incorporate updates or mutations, 5) the Query Reasoning Engine performing calculations according to the specific patient, situation and CKG, and deconstructing the updates and mutations so as to effect updates and mutations in external software systems; and 6) the Query Reasoning Engine storing, as determined by the CKG, portions of query responses updates, and mutations including new condensers, health situations and flows, as a case history graph in the KSS for later re-use.

FIG. 1C further illustrates the Query Reasoning Engine system, comprising modules of at least a Condenser Engine (CE) and a Dynamic Calling Engine (DCE), with a preferred embodiment also incorporating modules of a Determination Engine (DE) and a Transformation Engine (TE).

FIG. 1D further illustrates the above method step 3) in a preferred embodiment comprising the steps of
a) An API Gateway Server receiving health queries and returning responses in one or more formats, thereby performing load balancing, adaptation and communicating with the Determination and/or Condenser Engines. In a preferred embodiment, available formats include one or more of GraphQL and OpenAPI, although it will be understood that there are many options;
b) the Determination Engine determining from the query the relevant patient context, CKG, health situation, health context, and location within the overall health flow;
c) the Determination Engine according to the CKG and health query determining the list of minimum necessary top level health concepts and reasonings which are subsequently passed to the Condenser Engine;
d) the Condenser Engine recursively determining the graph of information that needs to be pulled from external systems, along with the corresponding valuator, and passing the results to the Transformation Engine or Dynamic Calling Engine for retrieval;
e) The Transformation Engine directly adapting the semantics of at least one of information, triggers and actions, between the Condenser Engine and the Dynamic Calling Engine; and
f) The Dynamic Calling Engine communicating with Healthcare Enterprise Software and the like via one or more adapters to match their specific communication method to retrieve the results;
g) The Transformation Engine adapting the returned results into health concepts for use by the Condenser Engine
h) The Condenser Engine's Resolver resolving downward and condensing upwards the health concepts utilizing the KSS, internal or external condenser modules, functions, reasoners or data according to the CKG. As it does so, storing the determinative factors from each health concept calculation in the KSS; and
i) the Condenser Engine condensing and including in its query response any requested chain of thought reasoning (including the determinative factors), potential actions as appropriate and context.

For illustrative purposes only, FIG. 1E provides an example of a representative but hypothetical chatbot chat when a large language model chatbot is the software application generating the health query to and formatting the response from the Query Reasoning Engine. For illustrative purposes only, FIG. 1F provides an example of a representative but hypothetical automatically constructed graphical response when the health query from a GUI-based application is to provide a full case status summary.

Determination Engine

As illustrated in FIG. 1C, the Determination Engine is comprised of Health Situation, Health Flow and Instantiator modules. The Health Situation is further comprised of: a) at least a Clinical Situation, which in turn is composed of at least a Case Status, and it may also incorporate a Case History, and Next Steps; and may also incorporate b) a Personal Situation; and c) an Economic Situations.

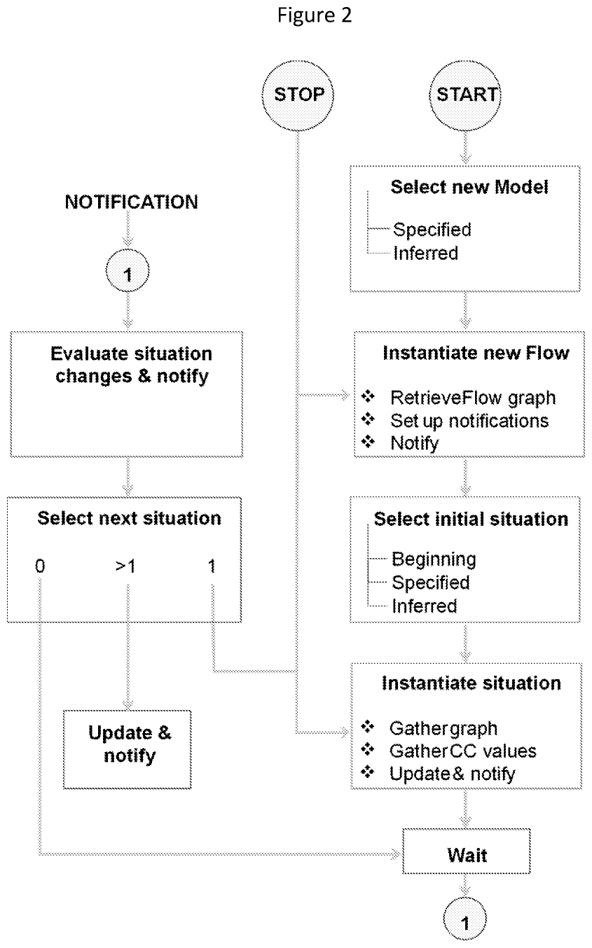

The method of the Determination Engine is illustrated in FIG. 2. The first step undertaken by the Determination Engine is to determine the appropriate portion of the CKG, which may be selected via the health query, or it may be inferred. There may be one or more CKGs in use for any given patient at any given time. By way of illustration, a urologic oncologist might utilize a guidelines-based CKG for prostate cancer with one patient, while for the same patient having gone to the ER with a chest pain, the system might infer for a cardiology nurse a cardiology illness script CKG. The same oncologist, for a different patient might use a bladder cancer CKG, while their staff might use a simplified hematuria CKG.

The next step is for the Instantiator to instantiate the flow associated with the CKG, which involves retrieving the graph of its definition, and setting up notifications requested by the health query for change of state, as well as to notify (including record its instantiation in the KSS). For illustrative purposes, in guidelines-based reasoning, instantiation may entail retrieving the graph of health states, expressions, flow paths and the like.

Subsequently the Determination Engine will select the situation, either the first one in the flow, as a result of selection by a health query, or by inference. Next, the Instantiator will instantiate the situation, which further entails gathering the graph of requisite health concepts and reasonings, passing the graph to the Condenser Engine to gather their state; and setting up notifications for values that are yet to be determined (either initially or awaiting update), or in error, and via health query upon change of state; and notifying (i.e. updating the KSS).

The Query Reasoning Engine may respond to notifications autonomously and, according to the CKG may effect updates and mutations; construct and execute health queries; store portions of health query results, updates and mutations in the KSS for later re-use; trigger notifications to external software applications; and the like.

At this point, if initially requested as part of a health query, or a subsequent health query can retrieve the situation or any portion thereof as it changes over time.

Subsequent to initial instantiation of the Situation, there are multiple paths to update and change the Situation, including but not limited to, each of which may cause a notification (including updating the KSS):
i. Health query updates, that instantiate new situations (for illustrative purposes, as just one example, when applications are adding a case whose information is not already available in the underlying healthcare enterprise software)
ii. Health query updates when next steps are chosen (for illustrative purposes, as just one example, when a physician chooses a course of action)
iii. By reasoners (for illustrative purposes, as just one example, a reasoner detects that the patient is about to go into cardiac arrest)
iv. Upon triggering by changes, timers, alarms, external systems and the like (for illustrative purposes, as just one example, a lab test result arrives)
v. By a Condenser (for illustrative purposes, as just one example, lack of availability of equipment in a timely manner creates a new path selection)
vi. And the like

As there are situation updates, the necessity to instantiate a new situation is evaluated, and the updates to health concepts within the CKG are recorded in the KSS. The results of the evaluation may determine that: there is not yet a need to instantiate a new situation; there may be only one potential new situation; or there may be more than one potential situation that requires an external health query update to select.

When there is a single next situation, it may be an endpoint of the Flow according to the current CKG, in which case the Flow is completed. Otherwise, the Instantiator may instantiate a new Situation, or a new Flow and Situation. For illustrative purposes, as one of many potential examples, a Hematuria Flow, upon the addition of a of a high Risk Stratification, may result in the instantiation of a Suspicion of Bladder Cancer Flow.

A “Full Case History” is constructed over time by adding changes to Health Situations, Flows, and condensers as well as other notifications to the case history graph.

Condenser Engine

As illustrated in FIG. 1C, the Condenser Engine is comprised of three components: the Condenser, Resolver and Actioneer components.

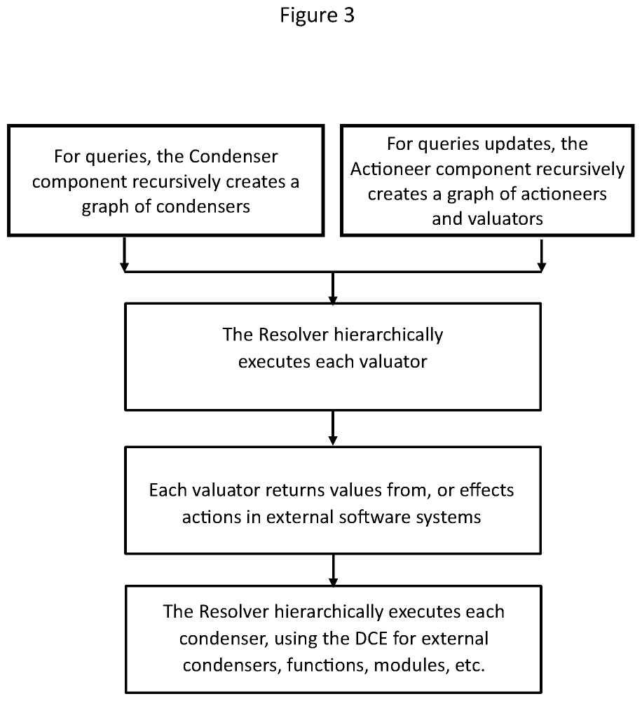

As illustrated in FIG. 3, the method of the Condenser Engine for health queries is that 1) the Condenser Engine recursively builds the graph, the condenser graph, of reasoning steps, known as condensers, as set forth in the CKG, required to populate the top-level graph received from the Determination Engine (or directly from the health query. This graph is passed to the Resolver, 2) the Resolver recursively walks the graph. Associated with each Condenser is a Valuator, if the Valuator determines that its corresponding information requires external software system access, the Resolver adds it to a Valuator Graph. 3) the Valuator Graph is passed to the Transformation Engine or directly to the Dynamic Calling Engine for information retrieval into the graph. 4) With the retrieved information, the Resolver recursively executes the Condenser Graph, utilizing internal or external condensers according to the CKG, and using the Dynamic Calling Engine to call external condensers (which may include reasoners, functions, ML models to populate a results graph that gets returned to the Determination Engine or to the health query directly.

Also illustrated in FIG. 3, the method of the Condenser Engine for health query updates is that 1) the Actioneer component builds the graph that deconstructs the updates into the lower level actions required to update external systems, including healthcare enterprise systems as necessary. This graph is passed to the Resolver, 2) the Resolver hierarchically walks the graph. Associated with each Actioneer is a Valuator function, which the Resolver executes to execute the graph of actions in the external systems.

The Condenser Engine is extensible, with the ability to plug in additional types of Condensers, Valuators and Actioneers.

Condensers

In a preferred embodiment, there are at least one of the following types of Condensers:
a) Semantic Qualifier condensers, which generalize characteristic values when the precise value isn't relevant. For example, young/middle-aged/old; acute/chronic; diffuse/localized; and the like
b) Clinical Syndromes condensers, which combine characteristics into a single medical concept, for example: “typical chest pain”=exertional chest pain, substernal of characteristic quality and duration, relieved with rest or nitroglycerin; “worsening chronic heart failure”=progressive systemic pulmonary congestion typically over days to weeks; and “acute hepatic failure”=jaundice, ascites, confusion, asterixis, TBS>2.5, INR>2.0; and the like.
c) Data condensers which gather one or more items of basic data from other sources, as may be found in a traditional medical knowledge graph or ontology.
d) Summary condensers which summarize which take more complex information and summarize it into a condensed representation, which may or may not include “determinative details,” for example: The summary of a risk stratification might be high, intermediate or low, and the determinative details may include the risk factors, such as smoking; family history of cancer, age; etc. in oncology; The summary of a cystoscopy result, might be normal, abnormal or indeterminate, and the determinative details may be characterized, in the case of an abnormal result, by tumor size, type and location; The summary of a urinalysis in the context of microhematuria might be infection or no infection, and the determinative details might be the value of RBC/HPF, WBC and NIT+LEU
e) Document condensers, which may include context-specific condensers for the various types of electronic health records, such as family histories, allergies, lab imaging or genetics tests, reports, notes, messages, etc.
f) Case Summary condenser”, which takes a full “Case Status and “Case History” and condenses it to only the subset of the graph that's relevant to a particular determination, such as the relevant case status, personal or economic information, the summarized clinical case history, locations in the overall health flow, health context. Other case summary condensers may be more specific, condensing the graph to a diagnosis, a next health step, or a qualification for a clinical trial.

Condensers are further defined by the full set of clinical, personal or economic concepts upon which they operate, which each may have a corresponding Condenser. Higher level medical concepts, such as “typical chest pain” are built from other medical concepts, such as “substernal of characteristic quality and duration”, which are further built of medical concepts, such as substernal, characteristic quality, etc. Condensers need not “operate” on a single value, or even a hierarchy of values, but rather they may operate on complex graphs. For example, “worsening chronic heart failure” operates on a complex graph of time-dependent sequences and values. Or, for example, a condenser for a CT scan, when operationalized via a machine learning-based condenser, might operate on the raw imaging data itself—or perhaps on the CT scan radiology report per se (perhaps in pdf form) to extract the summary and summary details. Condensers also may have associated roles and responsibilities, requisite skill sets equipment and facilities, and the like.

In FIG. 3, step 1) the Condenser module builds the graph of condensers, starting with those transmitted to it by the Determination Engine, and recursively builds a graph by determining the concepts upon which those operate, then determining the determined concepts' Condensers and adding them to the graph, and so on in turn.

Actioneers

Actioneers are a type of Condenser. They expand condensed choices and health query updates into external occurrences, such as adding a note, triggering an order set, scheduling a procedure, putting a patient on a well-known course of treatment and the like. As with other condensers, actioneers are further defined by the full set of concepts and actions upon which they operate.

Actioneers may have a Rationale, which may be comprised of “determinative” items leading to a specific set of potential choices; “indicative” items used in making specific recommendations; and explanatory items identifying the specific logic or more complex selection-making.

Valuators

Associated with each Condenser and Actioneer are Valuators. Valuators are defined by at least one of:
a) The data types and allowable values of the concepts and condenser they are associated with, for example Single valued: e.g. HbA1c ranges from 3% to 150%; Multi-valued: e.g. low, medium, high; Multi-value, multi-type: e.g. Lachman test; Translation grade: 1+, 2+, 3+, or <5 mm, 5-10 mm, >10 mm; and the like.
b) A state, with values including ready; pending; completed; and stale (which indicates the value is no longer valid); and each of the states has one or more time stamps associated with it for example, the date and time of the CT, or the date and time that the state of condenser changed.
c) A time base, with values including at least one of specific times; durations, which may include durations of validity; Trends, such as growing/shrinking (over time); rising/decreasing (over time); Periodicity and the like.

Valuators incorporate a function executed by the Resolver to determine its value, utilizing the Transformation Engine and Dynamic Calling Engine. For illustrative purposes, that may include being populated from an EHR, LIS, Imaging system; CRM; workflow or other healthcare software; being populated automatically via computer algorithm (including AI algorithm or reasoner)—perhaps including the data source like an image; a data lake; a live stream of medical data, etc.; being determined interactively, or via manual entry. Similarly, for illustrative purposes, Valuators of Actioneers may populate notes in the EHR; trigger workflows and order sets; trigger email notifications to patients; etc.

Transformation Engine

As illustrated in FIG. 1C, the Transformation Engine is composed of modules, an Information module and in a preferred embodiment a Triggers module and an Actions module. The Transformation Engine is responsible for directly mediating between the semantics of types in Valuators, and those in external systems (i.e. between internal and canonical, native or direct types). It incorporates a transformation ontology capable of mediating among: one-to-one; one-to-many; custom mappings; classification hierarchies; temporal; logical (unary, binary, extended). Transformation ontologies and approaches in healthcare are described in the literature (e.g. KDOM, OntoImportSuite, etc.).

Dynamic Calling Engine

The Dynamic Calling Engine is responsible for retrieving the information, setting up trigger notifications and effecting actions in external systems, as well as executing external condenser modules. It accepts calls from the Resolver or the Transformation Engine and utilizes the appropriate Adapter to execute the call in an external system which uses a different calling style or mechanism. Dynamic Calling Engines are described in the literature.

Tools

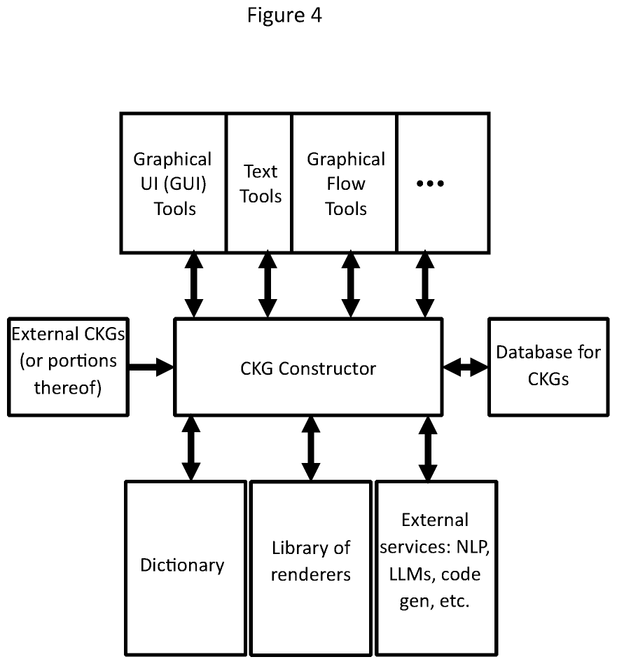

As illustrated in FIG. 1A, the current disclosure also provides a method of constructing CKGs wherein different clinicians of normal skill in the medical arts can construct portions of the CKG, the portions which later can be combined to create full CKGs. As illustrated in FIG. 4 the system component further comprises: 1) a CKG Constructor, 2) a database to store the CKG under development, along with elements of other CKGs; 3) a dictionary to hold traditional knowledge graphs and ontologies; 4) an import module to load CKGs developed separately; 5) a variety of tools that provide different styles and capabilities for constructing CKGs; 6) a library of Renderers for Condensers; and 6) External services including at least one of but not limited to natural language model libraries, generative language and generative code services.

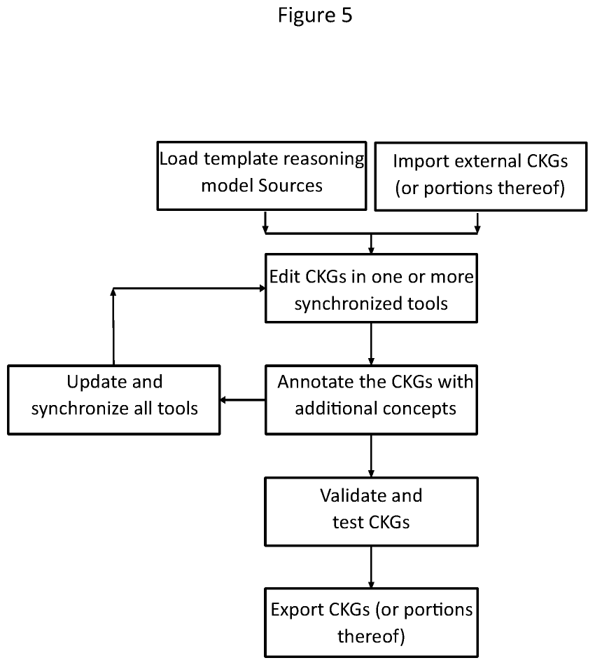

FIG. 5 illustrates the corresponding method, comprising the steps of 1) selecting from among supported template reasoning model Sources, which may grow over time but which include exemplar case presentations, health flow models such as diagnostic or care flowcharts, illness script diagnostic reasoning, adjacent disease differentiator reasoning and the like; 2) optionally loading all or portions of existing CKGs constructed by related or unrelated parties; 3) edit the CKG in one or more synchronized tools in any sequence or order to create exemplar health situations (comprised of clinical, personal and health situations, clinical situations further comprised of case status, history, next steps) and health flows, matched to graphs of condensers and actioners. The tools may include: written case examples analyzed by natural language interface-based tools; graphical flow building tools; graphical user interface building tools; checklist building tools; risk stratifier building tools, illness script building tools, differential building tools, and external tools as may be added from time to time; 4) annotating the resulting CKGs with additional clinical, personal or economic condensers; 5) validate and test the CKG; and 6) optionally export the CKGs for use by related or unrelated parties.

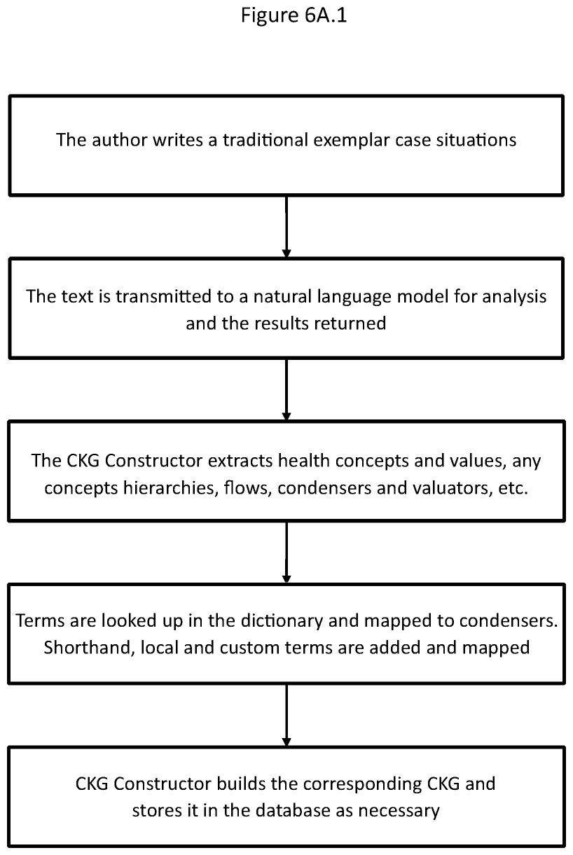

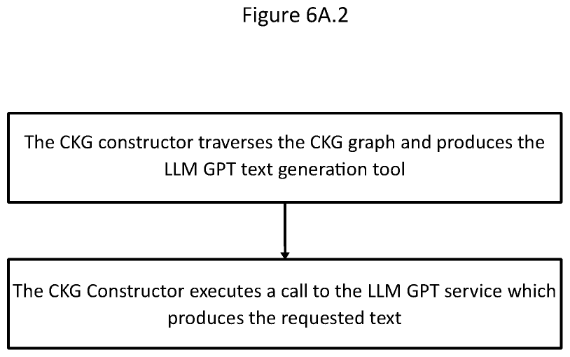

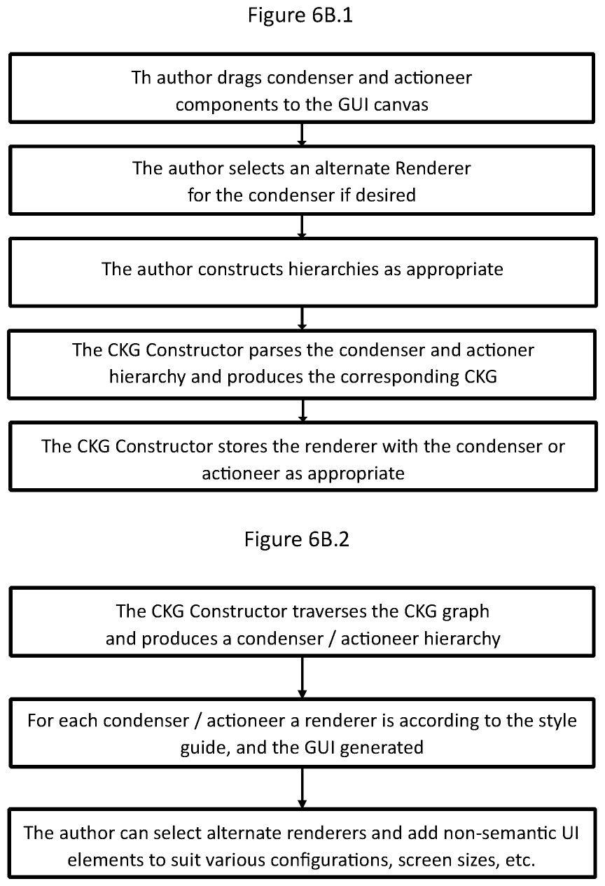

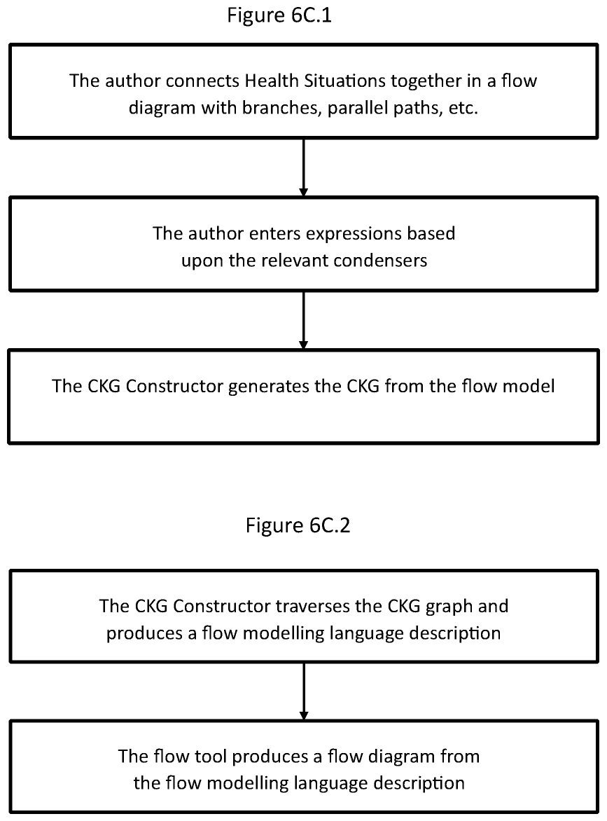

FIGS. 6A, B and C further illustrate the method step 3 above for written, GUI and flow diagram tools respectively. In FIG. 6A.1 the steps for specifying the health situation and flow via traditional written textual case description include: 1) the author either writes the exemplar case situation as normal, which further may consist of defining Condensers and Valuators using natural language, or loads a document containing a Health Flow model (such as an clinical guideline); 2) natural language model is used to parse and analyze the text; 3) the CKG Constructor parses the results of the natural language model analysis to extract the health concepts and values, Condensers and Valuators, any hierarchy or, any potential next steps with expressions and the like; 4) terms are looked up in the dictionary, which is a traditional knowledge graph and ontology populated with basic medical terminology such as SNOMED, LOINC, CPT along with the mappings among them and any existing corresponding condenser identified (i.e. creating a computational knowledge graph from a traditional one). Any terms introduced in the entered text such as shorthand or local terminology may be added; 5) CKG Constructor builds the corresponding CKG graph and stores it in the database as necessary. In FIG. 6A.2 the steps for generating the text description from a CKG are: 1) the CKG Constructor traversing the CKG graph and producing the corresponding graph for a large language model generative pre-trained transformer (LLM GPT) component; 2) the CKG Constructor executing an API call to the LLM GPT, which in turn produces the requisite text.

In a preferred embodiment of the current disclosure, from a textual description of a Condenser and Valuator, the LLM GPT may generate computer code for external condensers and valuators.

In FIG. 6B.1 steps for specifying the health situation and flow via a GUI Design Tool include 1) the author dragging Condenser and Actioneer components to the GUI design canvas, adding labels where appropriate; 2) The author choosing other than the default Renderer for the Condenser or Actioneer from the library, if desired; 3) the author constructs hierarchies as appropriate; 4) the CKG Constructor parses the Condenser and Actioneers, along with their hierarchies, and produces the corresponding CKG; and 5) as appropriate, the CKG Constructor includes the Renderer for the corresponding constructed Condenser in the CKG.

In FIG. 6B.2, the steps for generating the GUI from a CKG are: 1) The CKG Constructor traverses the CKG Graph and produces the GUI Condenser/Actioneer hierarchy; 2) for each Condenser/Actioneer, a Renderer is chosen according to a style guide; and 3) the Author can select alternative Renderers as desired.

In a preferred embodiment of the present disclosure, the GUI tool may add additional non-semantic user interface elements, as well as different non-semantic visual configurations and hierarchies to suit different size screens, and the like.

In a preferred embodiment, a module which computes and executes the steps of FIG. 6B.2 may be utilized by GUI applications which generate health queries to the system of the current disclosure, in order to produce a GUI to display responses either statically beforehand or dynamically to match the specific health query results. In a preferred embodiment, health queries may return a graph of Renderers and Valuators with their results, enabling software system to automatically constructs a graphical user interface in response to health query results, according to the CKG.

In FIG. 6C.1 steps for specifying the health situation and flow via a Graphical Flow Tool in the case of a guidelines-based CKG include 1) connecting Health Situations together in a flow diagram with branches, parallel paths, and other options typical in process flow modelling, 2) entering expressions based upon the corresponding condenser, and 3) producing the corresponding CKG. In FIG. 6C.2 the steps for generating the Flow Diagram from a CKG are: 1) the CKG Constructor traversing the CKG graph and producing a Flow modelling language description; and 2) the Flow tool utilizing the Flow modelling language description to produce the Flow diagram.

In a preferred embodiment of the current disclosure, the graphics style of the Flow diagram may correspond to that used by the corresponding health guideline.

FIG. 5 method step 5) when undertaken is further comprised of at least one of: 1) an automated test suite to build and automate test cases; 2) a trace tool to track and verify execution paths; 3) a coverage tool to assess test coverage; 3) a completeness check to compare the CKG to a corpus of medical knowledge in a knowledge graph; a version tracking tool to track and note CKG versions. While well known in software, they are novel in their application to CKGs.

Validated Data

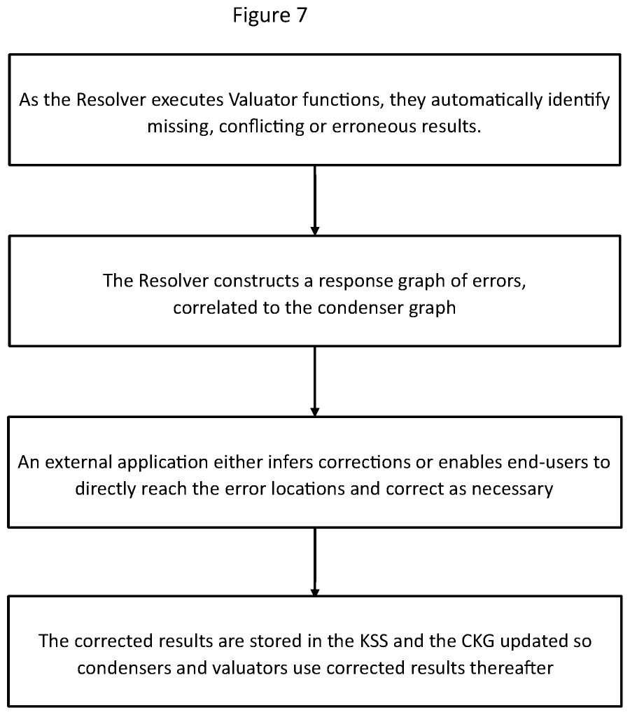

The current disclosure provides a method of producing validated, trustworthy health concept values from enterprise healthcare systems, during FIG. 1B step 4 above, and as further illustrated in FIG. 7, comprising the steps of i) As the Resolver executes each Valuator function, the Valuator automatically identifying missing, conflicting, or erroneous health information; ii) the Resolver constructing a response graph containing these results which may be correlated to the Condenser graph; iii) an external application enabling end-users via a graphical user interface or automated systems navigate the Condenser Graph to reach the error locations identified in the error response graph; and iv) storing health information corrections in the KSS for later use in lieu of missing or duplicate or erroneous information by updating the CKG so in future Condensers and Valuators access the corrected results.

Clean Data

It will be understood by those skilled in the art that health queries may span patients and that results may be exported in order to produce clean data sets for use with typical analytics or machine learning software.

Economics

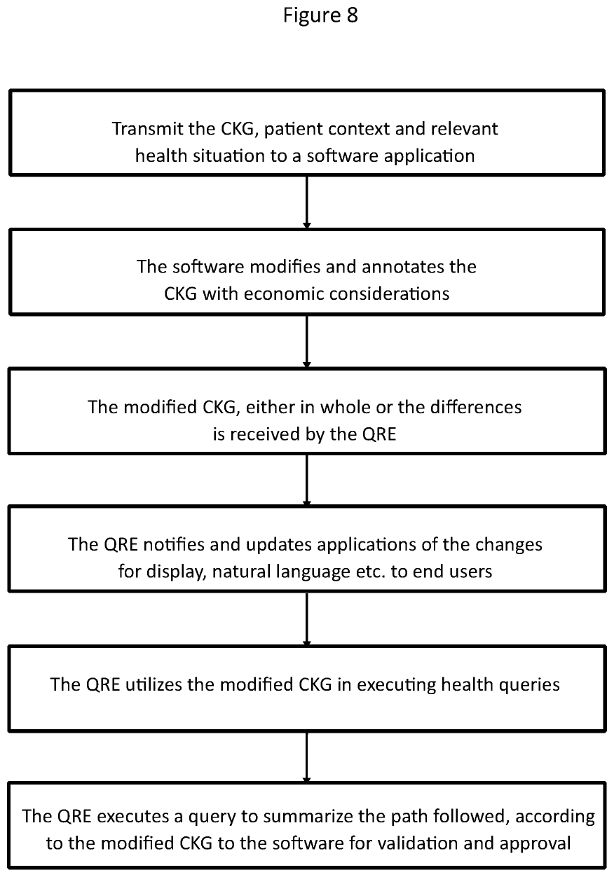

As Illustrated in FIG. 8, the current disclosure also provides a method facilitate economic reasoning, comprising the steps of 1) transmitting the CKG, patient context and relevant health situation to an external software application, by way of example to software operated by healthcare insurance providers or by the finance department of healthcare providers; 2) the external software annotating the CKG with additional economic considerations (such as patient out of pocket costs, expected future costs, RVU and the like), or modifying the CKG (such to add or remove prerequisites or covered branches); and 3) receiving from an external software application a modified CKG, either in total or as the differences between the CKGs; 4) the Query Reasoning Engine notifying and updating applications of the changes for display to users; 5) the Query Reasoning Engine utilizing the modified model in the process of executing health queries; and 6) transferring to the external software the reasoning path followed, according to the modified CKG for validation and approval.

In a preferred embodiment of the disclosure, the Query Reasoning Engine will, when queried, return in response to a health query the differences between the CKGs, or return the modified CKG. In a preferred embodiment of the disclosure, the Query Reasoning Engine will validate to the external software that the modified CKG was followed.

What is claimed is:

1. A system, comprising:
a non-transitory computer readable storage medium storing an executable program;
and a processor executing the executable program to cause the processor to:
(1) load a CKG
(2) accept a health query; and
(3) utilize the CKG in the process of executing the health query.

2. The system of claim 1, wherein the processor executing the executable program to cause the processor to act, according to the reasoning model, at least one of:
(1) returning results of the health query;
(2) effecting updates and mutations in external software systems;
(3) accessing data in the external software systems and processing the data;
(4) storing portions of the health query results for later re-use; and
(5) storing portions of the updates and mutations for later re-use.

3. The system of claim 2, wherein the processor executing the executable program to cause the processor to load one or more CKGs.

4. The system of claim 3, wherein the processor executing the executable program to cause the processor to utilize CKGs consisting of health situations and health flows matched to graphs of condensers and actioneers.

5. The system of claim 4, wherein the processor executing the executable program to cause the processor to accept the health queries from at least one of:
(1) a software system which uses natural language model to interact with its users;
(2) a software system which uses a graphical user interface to interact with its users;
(3) a software system which uses an audio interface to interact with its users;
(4) a software system which has no user interaction; and
(5) a software system wherein the query was not triggered by direct user interaction.

6. The system of claim 5, wherein the software system automatically constructs a graphical user interface in response to the health query results, according to the CKG.

7. The system of claim 4, wherein the processor executing the executable program to cause the processor to respond to notifications and autonomously at least one of, according to the CKG:
(1) construct and execute health queries;
(2) effect updates and mutations;
(3) store portions of health query results for later re-use; and
(4) store portions of updates and mutations for later re-use.

8. The system of claim 7, wherein the processor executing the executable program to cause the processor to return a case summary including two or more of:
(1) relevant case status information;
(2) summarized clinical case history;
(3) relevant personal information;
(4) relevant economic information;
(5) location in the overall health flow; and
(6) health context.

9. The system of claim 8, wherein the processor executing the executable program to cause the processor to include in its query response at least one determinative factor.

10. The system of claim 9, wherein the processor executing the executable program to cause the processor to utilize external condenser modules according to the CKG.

11. The system of claim 9, wherein the processor executing the executable program to cause the processor to act at least one of:
(1) automatically identifying missing or conflicting or erroneous health information; and
(2) storing health information corrections for later use in lieu of missing or duplicate or erroneous information.

12. The system of claim 9, wherein the processor executing the executable program to cause the processor to act at least one of:
(1) transmitting one of the CKGs, a patient context and a health situation to a software application;
(2) receiving from the software application a modified CKG; and
(3) utilizing the modified model in the process of executing the health queries.

13. The system of claim 9, wherein the processor executing the executable program to cause the processor to at least one of:
(1) return in response to the health query the differences between the CKGs; and
(2) return in response to the health query the modified CKG.

14. A system for creating and editing reasoning models, the system comprising:
a non-transitory computer readable storage medium storing an executable program;
and a processor executing the executable program to cause the processor to:
(1) load all or portions of one or more of existing CKGs or templates;
(2) edit the CKG in one or more synchronized tools to create exemplar health situations;
(3) test the CKG; and
(4) export the CKG.

15. The system of claim 14, wherein the processor executing the executable program to cause the processor to edit the CKG, wherein the CKG consists of health situations and health flows matched to graphs condensers and actioneers.

16. The system of claim 14, wherein the processor executing the executable program to cause the processor to edit the CKG utilizing at least one of:
(1) a natural language interface-based tool;
(2) a graphical user interface building tool;
(3) a health flow building tool;
(4) a checklist building tool;
(5) an illness script building tool;
(6) a differential building tool;
(7) a risk stratifier building tool;
(8) a natural language tool that loads documents containing Health Flow models; and
(9) an external tool.

17. The system of claim 16, wherein the processor executing the executable program to cause the processor to generate code for external condensers.
